(19) United States
(12) Patent Application Publication
(10) Pub. No.: US 2023/0277299 A1
(43) Pub. Date: Sep. 7, 2023

(54) NOVEL IMPLANT(S) AND SYSTEM(S) FOR IMPLANTATION AND FILLING

(71) Applicant: Doohi LEE, Plano, TX (US)

(72) Inventor: Doohi LEE, Plano, TX (US)

(21) Appl. No.: 18/197,140

(22) Filed: May 15, 2023

Related U.S. Application Data
(63) Continuation-in-part of application No. 17/671,260, filed on Feb. 14, 2022.

(51) Int. Cl. A61F 2/12; A61F 2/00

(52) U.S. Cl. CPC A61F 2/12; A61F 2/0059; A61F 2250/0003

(57) ABSTRACT

An implant including a shell and a valve. The valve preferably is larger than a first type of valve configured for introducing saline into the shell and smaller than a second type of valve configured for introducing silicone into the shell. The implant also preferably includes a filling for the shell. The filing may be a material other than saline or silicone, for example hyaluronic acid. The valve preferably is configured for introducing hyaluronic acid into the shell. The valve may be a single-use valve resistant against leakage of the hyaluronic acid after use or a multi-use valve resistant against leakage of the hyaluronic acid after each use. The shell preferably is made of or includes silicone. Also, associated system used to fill such an implant after implantation.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND

The present disclosure generally relates to novel implant(s), particularly ones filled with other than saline or silicone, and an associated system(s) for implantation and filling of such implant(s).

### SUMMARY

Aspects of the subject technology include an implant. The implant includes a shell and a valve. The valve preferably is larger than a first type of valve configured for introducing saline into the shell and smaller than a second type of valve configured for introducing silicone into the shell. The implant also preferably includes a filling for the shell. The filing may be a material other than saline or silicone, for example hyaluronic acid. The valve preferably is configured for introducing hyaluronic acid into the shell. The valve may be a single-use valve resistant against leakage of the hyaluronic acid after use or a multi-use valve resistant against leakage of the hyaluronic acid after each use. The shell preferably is made of or includes silicone.

Other aspects of the subject technology include a system for filling an implant after implantation. The system preferably includes a tube configured for attaching to a valve of the implant, with the tube larger than a first type of tube used to transfer saline and smaller than a second type of tube used to transfer silicone. The system also preferably includes a delivery device configured to fill the implant through the tube and the valve with a filling comprising a material other than saline or silicone, for example hyaluronic acid. The delivery device may be an infusion pump, a syringe, or some other device suitable for introducing the filling material into an implant. Again, the valve may be a single-use valve resistant against leakage of the hyaluronic acid after use or a multi-use valve resistant against leakage of the hyaluronic acid after each use. The implant preferably includes a shell, for example made of or including silicone.

This brief summary has been provided so that the nature of the invention may be understood quickly. Additional steps and/or different steps than those set forth in this summary may be used. A more complete understanding of the invention may be obtained by reference to the following description in connection with the attached drawings.

### BRIEF DESCRIPTION OF THE DRAWINGS

FIG. 1 illustrates an implant according to aspects of the subject technology.

FIG. 2 illustrates a system for filling an implant after implantation according to aspects of the subject technology.

### DETAILED DESCRIPTION

Overview

The following paragraphs [0009] to [0022] provide an overview of aspects of the subject technology from the inventor Doctor Doohi Lee's own professional medical perspective. The subject technology is not limited to this overview. Additional aspects are discussed below under Embodiments.

Breast augmentation is one of the most common and requested surgery in the field of cosmetic surgery. Many techniques of breast augmentation exist, from injecting fat and other materials (in the past with often bad results), to placing implants under the breasts. The most common techniques are augmentation with silicone implants filled with either silicone or sterile saline. The techniques both result in enlarging the space beneath the breasts to create enlarged or augmented breasts.

Through the years, many techniques have been developed to introduce the implants to the breasts. The spaces in the chest are either immediately deep to the breasts (submammary) or deep to the pectoralis muscle (submuscular).

To enter these spaces, several surgical access approaches exist. The most common is the inframammary, which is to enter from the fold of space between the breasts and the chest. Inframammary augmentation can result in a scar under the breasts. Nearly as common approach is through the areola, where a circular incision around the areola is made to enter the submammary or submuscular space. The third approach is the axillary approach where an incision is made in the axilla and the access is created to enter the desired space.

The least common approach (technique) is the trans-umbilical approach, where the access is created through an incision at a patient's umbilicus or bellybutton. The full name of this approach is the “trans-umbilical breast augmentation” or TUBA. The TUBA technique is rarely used by surgeons (not many are trained in this technique) because of technical difficulty and the limitation to saline implants only. The chief advantage of this technique is that the scar is hidden in the umbilicus and the breasts are completely scar free. This approach has additional hidden advantage in that some patients have problems with keloid scar formation with the other approaches, and many have discovered this problem after their breast augmentation surgery with those approaches. They then had to deal with keloid(s) under their breasts. Formation of such keloid scars is far less likely with the TUBA approach.

One disadvantage of the TUBA approach to date is limitation to saline implants. The subject technology attempts to address this disadvantage.

The primary difference between saline and silicone filled implants is the texture and the “feel” of the implants. The silicone filled ones have various firmness to them, depending on the complexity or the density of the silicone and many people perceive that silicone implants more realistically replicate the feel of natural breasts. This perception is of course highly subjective and varies widely between individuals.

On the other hand, the saline filled implants may be perceived to feel more like “water balloons” because essentially that is what they are. Depending on how much saline is used to fill the implants, the texture can vary from floppy (under filled water balloon) to rock hard (over filled water balloon). Often, depending widely on surgeon's techniques, the feel and texture of the saline implants are quite different from silicone implants. For these reasons and others, the silicone implants are much more popular and more often selected for breast augmentations.

The inventor of the subject technology, Dr. Doohi Lee, has practiced and performed cosmetic surgery for many years and is an expert in the TUBA technique of breast augmentation. Even though he feels that TUBA is an ideal technique for many women, the lack of option to augment with silicone implants has relegated TUBA to more of an academic discussion and option. Goal(s) and benefit(s) of the subject technology include changing this perception and bring the TUBA technique to be much more mainstream by creating an implant that can be used in the TUBA technique but filled with a material other than saline. Other goals and benefits may be realized using the subject technology.

Because of many factors, FDA limitations being a dominant one, as well as previous debate about deleterious effects of silicone in the body, the possibility of introducing silicone through the umbilicus is an unlikely occurrence. Thus, the inventor has created a novel approach using a material that is already FDA approved for cosmetic use and already has proven safety record in the human body: hyaluronic acid (“HA”).

The HA is the chief material for the vast majority of facial fillers placed throughout the world, from cheeks, chin, lips, noses, and many other aspects. HA is already a part of the human body; HA is ubiquitous to any connective tissue including muscles, tendons, ligaments, etc. After placement, HA degrades with time and is completely absorbed by the body, very much like saline. For example, when a saline implant ruptures, the saline is expelled and immediately absorbed by the body and only the silicone shell is left.

Another advantage is that HA has various textures and firmness depending on the complexity of its manufacture, from very thin to very thick and robust firmness. HA filled implants may not rival that of the silicone filled implants, but certainly will have much more substantial and denser characteristics than saline filled implants. Another factor is that because of its semi-liquid characteristics, HA can be instilled through a delivery system suitable for TUBA techniques.

For the purposes of breast augmentation, a silicone shell for an implant can be placed into a desired space (submammary or submuscular) through a tube via an incision at a patient's umbilicus or bellybutton (i.e., TUBA approach). Instead of saline, HA can be delivered into the implant. It is the belief of the inventor that the present silicone shells for saline can more than adequately accommodate the HA and hold this filling in place as the implant. Just as saline does not escape the silicone shell, neither would HA. But the results would be a much firmer and more natural feel of the breasts after the HAn implantation.

Apart from using an existing FDA material in a novel fashion, further aspects of the subject technology include a system for delivery of HA through the tube used for the TUBA approach and a suitable valve for an implant. Present delivery system may be adequate for the saline, which is liquid and does not require a large bore. However, HA is thicker and would require a more robust delivery system with a larger delivery tube. Also filing an implant with HA using a device such as a large bore syringe (e.g., 60 ml.) attached to the tube would be extremely tiring. Use of an infusion pump, possibly configured to accept pre-filled bags of HA holding a particular quantity to achieve a desired augmentation, may be used to fill an implant through a tube after positioning.

In conclusion of this overview, goal(s) and benefit(s) of the subject technology is to provide a new type of breast augmentation filler by using an existing FDA approved material, namely HA. The HA may be introduced by an elegant surgical technique that leaves no scars on a patient's breast. The inventor believes that HA is an ideal material to bridge the gap between saline and silicone, resulting in a new type of implant experience that will result in a natural feel of the breasts while hopefully optimizing aesthetic outcomes and reducing potentially deleterious consequences such as keloids and scarring.

### EMBODIMENTS

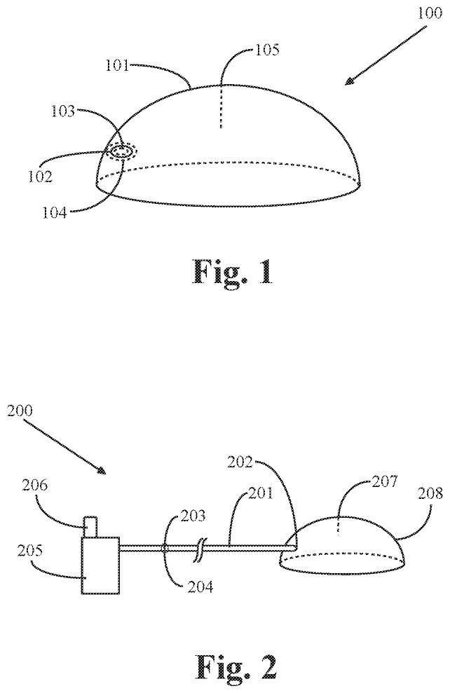

FIG. 1 illustrates an implant according to aspects of the subject technology. Implant 101 includes shell 102 and valve 103. In some aspects, implant 101 may be a breast implant. In other aspects, implant 101 may be any kind of implant, for example but not limited to face, chest, abdomen, buttock, arm, leg, etc., etc. implant(s). The various associated body cavity and/or cavities may be augmented by expandable silicone shell implants whose materials are already in use, but their shape and/or shapes will vary according to the relevant body cavity and/or cavities.

The valve preferably is larger than size 103 of a first type of valve configured for introducing saline into the shell and smaller than size 104 of a second type of valve configured for introducing silicone into the shell. The implant also preferably includes filling 104 for the shell. The filing may be a material other than saline or silicone, for example hyaluronic acid. The valve preferably is configured for introducing hyaluronic acid into the shell. The valve may be a single-use valve resistant against leakage of the hyaluronic acid after use or a multi-use valve resistant against leakage of the hyaluronic acid after each use. The shell preferably is made of or includes silicone.

FIG. 2 illustrates a system for filling an implant after implantation according to aspects of the subject technology. System 200 for filling an implant after implantation is shown. The system preferably includes tube 201 configured for attaching to valve 202 of an implant. The break shown in FIG. 2 represents that the size of tube 201 is not necessarily to scale with respect to the shown implant.

Tube 201 preferably has a larger size 203 than a first type of tube used to transfer saline and a smaller size 204 than a second type of tube used to transfer silicone. The system also preferably includes a delivery device configured to fill the implant through the tube and the valve with a filling comprising a material other than saline or silicone, for example hyaluronic acid. The delivery device may be an infusion pump 205, a syringe, or some other device suitable for introducing the filling material into an implant through the tube. Use of an infusion pump may reduce a surgeon's fatigue during a TUBA operation, for example as opposed to using even a large bore syringe. Infusion pump 205 may be configured to accept pre-filled bags of HA holding a particular quantity to achieve a desired level of augmentation. Use of pre-filled bags may help preserve sterility and facilitate filling an implant with a specific desired about of hyaluronic acid.

Again, valve 202 may be a single-use valve resistant against leakage of the hyaluronic acid after use or a multi-use valve resistant against leakage of the hyaluronic acid after each use. The implant preferably includes shell 207, for example made of or including silicone, which is filled with material 208 other than saline or silicone. This material preferably is hyaluronic acid.

Some possible goal(s) and benefit(s) of the shown embodiments are discussed above under Overview. Other goal(s) and benefit(s) may be realized.

The invention is in no way limited to the specifics of any particular aspects (e.g., embodiments, elements, steps, and/or examples) disclosed herein. For example, the terms “aspect,” “example,” “preferably,” “alternatively,” and the like denote features that may be preferable but not essential to include in some embodiments of the invention. Details illustrated or disclosed with respect to any one aspect of the invention may be used with other aspects of the invention. Additional elements and/or steps may be added to various aspects of the invention and/or some disclosed elements and/or steps may be subtracted from various aspects of the invention without departing from the scope of the invention. Singular elements/steps imply plural elements/steps and vice versa. Some steps may be performed serially, in parallel, in a pipelined manner, or in different orders than disclosed herein. Many other variations are possible which remain within the content, scope, and spirit of the invention, and these variations would become clear to those skilled in the art after perusal of this application.

What is claimed is:

1. An implant, comprising:
a shell; and
a valve larger than a first type of valve configured for introducing saline into the shell and smaller than a second type of valve configured for introducing silicone into the shell.

2. The implant as in claim 1, further comprising a filling for the shell, the filing comprising a material other than saline or silicone.

3. The implant as in claim 2, wherein the filing comprises hyaluronic acid.

4. The implant as in claim 1, wherein the valve is configured for introducing hyaluronic acid into the shell.

5. The implant as in claim 4, wherein the valve comprises a single-use valve resistant against leakage of the hyaluronic acid after use.

6. The implant as in claim 4, wherein the valve comprises a multi-use valve resistant against leakage of the hyaluronic acid after each use.

7. The implant as in claim 1, wherein the shell comprises silicone.

8. A system for filling an implant after implantation, comprising:
a tube configured for attaching to a valve of the implant, the tube larger than a first type of tube used to transfer saline and smaller than a second type of tube used to transfer silicone; and
a delivery device configured to fill the implant through the tube and the valve with a filling comprising a material other than saline or silicone.

9. The system as in claim 8, wherein the delivery device comprises an infusion pump.

10. The system as in claim 8, wherein the filling comprises hyaluronic acid.

11. The system as in claim 10, wherein the valve comprises a single-use valve resistant against leakage of the hyaluronic acid after use.

12. The system as in claim 10, wherein the valve comprises a multi-use valve resistant against leakage of the hyaluronic acid after each use.

13. The system as in claim 8, wherein the implant further comprises a shell.

14. The system as in claim 13, wherein the shell comprises silicone.

15. An implant, comprising:
a shell; and
a filling for the shell comprising a material other than saline or silicone.

16. The implant as in claim 15, wherein the filling comprises hyaluronic acid.

17. The implant as in claim 15, further comprising a valve for introducing the material other than saline or silicone into the shell, the valve larger than a first type of valve configured for introducing saline into the shell and smaller than a second type of valve configured for introducing silicone into the shell.

18. The implant as in claim 17, wherein the valve comprises a single-use valve resistant against leakage of hyaluronic acid after use.

19. The implant as in claim 17, wherein the valve comprises a multi-use valve resistant against leakage of hyaluronic acid after each use.

20. The implant as in claim 15, wherein the shell comprises silicone.
